(19) United States
(12) Patent Application Publication
(10) Pub. No.: US 2013/0323721 A1
(43) Pub. Date: Dec. 5, 2013

(54) HEPATOCYTES FOR IN VITRO GENOTOXICITY TESTS

(71) Applicants: Adrianus J.C.M. Braspenning, Heidelberg (DE); Stefan Heinz, Darmstadt (DE); Astrid Nörenberg, Glashutten (DE); Nicola Hewitt, Erzhausen (DE); Jan-Heiner Küpper, Grosskoschen (DE)

(72) Inventors: Adrianus J.C.M. Braspenning, Heidelberg (DE); Stefan Heinz, Darmstadt (DE); Astrid Nörenberg, Glashutten (DE); Nicola Hewitt, Erzhausen (DE); Jan-Heiner Küpper, Grosskoschen (DE)

(73) Assignee: MEDICYTE GMBH, Heidelberg (DE)

(21) Appl. No.: 13/877,581

(22) PCT Filed: Oct. 4, 2011
(86) PCT No.: PCT/EP2011/067295

§ 371 Date: Aug. 16, 2013

(30) Foreign Application Priority Data
Oct. 4, 2010 (DE) 10 2010 041 958.3

(51) Int. Cl. G01N 33/50

(52) U.S. Cl. CPC G01N 33/5067
USPC 435/6.1

(57) ABSTRACT

The invention relates to a method for carrying out genotoxicity tests of chemical, biological and physical active substances or agents with the aid of cell culture systems of proliferating physiologically active liver cells.

The invention relates to a method for carrying out genotoxicity tests of chemical, biological and physical active substances or agents with the aid of cell culture systems of proliferating physiologically active liver cells. The method is particularly suited for the genotoxic testing of both known and new drugs and active substances as well as combinations thereof in humans and animals. Moreover, it is suited to test chemicals or biological active substances in foods, cosmetics, textiles, materials and other substances for the genotoxic effect thereof in humans and animals. In many sectors of industry such as the pharmaceutical, cosmetics, food and chemicals industries, for example, new chemicals and/or biological active substances and combinations thereof are continually developed, the potential harmful effects thereof to people's healthy are generally unknown. Here, entirely different effects may occur in humans or animals. Drugs, chemicals or biological active substances can, for example, also develop undesirable side effects such as liver damage, damage to the mycoardium, neurotoxicity or teratogenicity, in addition to the desired effect within the meaning of the therapy. In the process, many cells of an organ may be lost, including degenerative organ disease, for example cardiac failure or liver damage. The cause of this toxicity can basically be due to all compartments and functions of a cell becoming damaged or being influenced, which is to say, for example, damage of the cell membrane, influence on physiological processes such as cell respiration, intracellular transport, signal transduction and gene expression, just to name a few examples. The invention relates to the direct or indirect action of agents on the DNA genetic makeup in human or animal cells and the suitable testing thereof, by means of so-called genotoxicity tests.

Providing suitable cells for testing is a medical and diagnostic challenge, in particular in the development of in vitro cell systems, including the related cell cultures.

As a result, a great need exists for establishing cell cultures/cell systems that are similar to human cells to the greatest extent possible, so that valid in vitro genotoxicity testing can be carried out.

Cell lines have become established in the prior art, which are cells that can reproduce without limitation on an appropriate culture medium and are immortal. In particular tumor cells or tumor-like cells are known, such as HeLa cells—the cervical cancer cell line, COS cells, HEK 293 cells—kidney, Chinese hamster ovary (CHO) cells, HEp-2—the human epithelial larynx carcinoma cell line and many more. The production of such cell lines is described in EP833934 (Crucell), for example Cell lines such as these are used, for example, for drug testing. However, the drawbacks of such cell lines are the genetic changes (such as point mutations, translocations of chromosome parts (rearrangements), an increase in the copy number of genes (gene amplification), and even changes in the sets of chromosomes (aneuploidy)) as well as the tumor properties due to lacking contact inhibition, whereby the cells are empowered into in vitro growth on soft agar substrates. Tumor cells additionally can grow an unlimited number of cell divisions that is due to immortalization. It is known that the cells of such cell lines gradually transform over the course of cultivation due to spontaneous mutations and can develop into a malignant cell population and are genetically unstable. Based on the inventors' findings, a critical threshold of accumulated mutations occurs in the culture after only approximately 60 cell divisions. These can be mutations, which lead to the activation of oncogenes or inactivation of tumor suppressor genes.

As a result, cells that can prevail in a cell population are those that exhibit increased cell division activity due to the accumulated mutations. This selection process corresponds to the precancerous condition in the development of tumors; additionally, cell lines that are commercially available usually have already undergone an unknown number of doubling processes, if they do not originate from malignant tumor cells to begin with.

Moreover, the following genotoxicity tests are described in the prior art: In the so-called Ames test (Ames et al., 1973a; Arnes et al., 1973b), bacteria that, due to mutation, for example point mutation in a gene, are no longer able to synthesize a particular amino acid (auxotrophic mutants) are applied to a culture medium (agar) that does not contain this amino acid. Because these bacteria are dependent on this amino acid for their continued existence, they would die off or would not be able to reproduce on this nutrient deficient medium. The bacteria are then exposed to the potential mutagenic substance, for example by placing a filter paper impregnated therewith on the culture medium. If so-called bacteria colonies form after subsequent incubation, individual bacteria have grown and have regained the ability to synthesize the amino acid in question. These are referred to as revertant colonies, in which a point mutation in a gene leading to auxotrophy has been reversed.

In the chromosome aberration test, the substances to be tested are incubated with cells. After a defined incubation period, chromosomal aberrations that occur are analyzed, for example by way of karyotype analyses. This method allows a plurality of chromosome aberrations to be rendered visible, such as, for example, the development of dicentric chromosomes, chromosomal breaks and sister chromatid exchanges (Morita et al., 1989).

Broschinski and colleagues report of the routine genotoxicity testing of 776 chemical substances, wherein a combination of the bacterial mutation test (Ames test) and the chromosome aberration test supplied the best sensitivity for detecting clastogenic agents (Broschinski et al., 1998).

Many agents only develop a genotoxic effect in an animal or human if these are chemically modified by liver enzymes. Differentiated hepatocytes, as they are present in an intact liver, in vivo have a variety of functions that are important for this biotransformation of substances in food, but also drugs or toxins (overview in Elaut et al., 2006). Phase I enzymes of the cytochrome P450 system are important for biotransformation. In humans, numerous isozymes are found, such as CYP1A1, CYP1A2, CYP2A6, CYP2B6, CYP2C8, CYP2C9, CYP2C19, CYP2D6, CYP2E1, CYP3A4, CYP3A5, CYP3A7, CYP4A11, which perform different functions. For some isozymes polymorphisms are known, which can be responsible for the individual variability in the toxic effect of drugs on the liver. CYP450 enzymes are oxidoreductases, which bring about oxidative degradation or metabolization of numerous substances, including pharmaceuticals.

In addition to the Phase I enzymes, Phase II enzymes exist, for example N-acetyltransferases [NATs] as well as UDP-glucoronyltransferases and sulfotransferases.

For assessing the potential liver toxicity of active substance candidates, and of chemicals in general, the functionality of the CYP450 systems, Phase II enzymes as well as other liver functions are of decisive importance. So as to take this circumstance into account, the Ames test is generally carried out in combination with a biotransformation of the substance to be tested using liver enzymes. For this purpose, the so-called S9 mix is generally used, which is a mixture of several liver enzymes so as to simulate a liver. The abbreviation “S9” refers to the supernatant and centrifugation of the liver cell extract at 9000 g. For example, De Flora et al. report that the substance phenacetin is only tested positively in the Ames test if an incubation of phenacetin is carried out with the S9 fraction of hamster liver (De Flora S. et al., 1985). This substance was transformed into a mutagenic form that can be detected in the Ames test solely by the enzymes that are active in liver cells. Another option for detecting DNA-damaging effects of agents is the so-called comet assay, also referred to as single cell gel electrophoresis (Singh et al., 1988). The principle of the comet assay is based on cells that are embedded in agarose undergoing lysis. The DNA of the cells is then exposed to an electric field. If the DNA was damaged by a substance or physical action, this can exit the nucleus and migrate toward the anode, while undamaged chromosomal DNA cannot do so. Under the UV microscope, the damaged cells, which previously were stained with fluorescent dyes such as ethidium bromide, are now observed as a tail of DNA pieces, giving them the appearance of a comet. The length of the tail of the comet is a measure of the DNA damage. The comet assay measures the level of DNA strand breaks, but provides no direct information about the underlying DNA damage.

A genotoxicity test that has been used increasingly over the past years is the so-called micronucleus test, by which cytogenetic changes can be detected significantly more easily and quickly than with the chromosome aberration test. Micronuclei contain parts of the nucleus which for different molecular reasons (damage to chromosomes due to clastogenic effects, damage of chromosome segregation due to aneugenic effects) are not incorporated in the daughter nuclei, but appear as chromatin particles in the cytoplasm. The number or frequency of occurrence of micronuclei is a measure of the genetic instability of cells. Cell division is generally necessary for new micronuclei to develop. In cells that, due to cytochalasin B, are inhibited in terms of mitosis, new micronuclei that were created by test treatments can be quantified in binuclear cells, while “old” micronuclei, which represent the background of the measurement, are determined in mononuclear cells (Fenech and Morley, 1985).

Since this technique was introduced, micronuclei are being increasingly analyzed as biological indicators of genotoxicity. This is primarily due to the fact that the evaluation of the micronucleus is relatively simple and fast, as compared to the evaluation of dicentric or otherwise aberrant chromosomes. Moreover, the automation of counting micronuclei is easier to do than for chromosomal aberrations or than is possible with the comet assay. The micronucleus test is frequently carried out in the Chinese hamster lung fibroblast V79 cell line or in human peripheral blood lymphocytes. In many examinations, different tests are usually combined, so as to obtain the most reliable information possible: for example Rossi and colleagues conduct an examination for potential genotoxicity of estrogens both with the Ames test, the chromosome aberration test and the micronucleus test (Rossi et al., 2007).

WO2004/034013 describes an alternative in vitro genotoxicity assay based on a special CHO cell line that contains human chromosome 11. This hybrid cell line expresses human CD59 protein, which presents itself on the cell surface. Mutations may result in a loss of this presentation on the surface, which can be detected by way of suitable immunological detection methods.

The problem of these tests is that to this day they are not sufficiently reliable, and moreover they are time-consuming and expensive. The pharmaceutical industry incurs high costs for genotoxicity assays. According to estimates of the Cambridge Healthtech Advances Life Sciences report of December 2004, imprecisely predicted genotoxicity accounts for approximately 30% of so-called drug failure costs. U.S. patent application US2008/0138820 A1 describes a micronucleus assay-based multiparameter genotoxicity assay. There, a construct, which constitutively expresses a fusion protein from a centromere protein using GFP, is introduced in a target cell line. This function allows micronuclei to be detected, which formed via an aneugenic mechanism. The nitroreductase coding sequence, the enzyme activity of which can be rendered detectable by way of a fluorescence conversion of the synthetic substrate CytoCy5S (GE Healthcare), is present on a second expression construct. If the nitroreductase is operably linked to a promoter that is activated by DNA damage (for example the GADD45a promoter), genotoxic effects that are clastogenic can be rendered detectable. By adding further cellular parameters, such as the proliferation index and cytotoxicity, an algorithm that is suitable for the respective cell system can be applied for a multiparameter analysis of potentially genotoxic substances.

However, there is a great need to use suitable cells for in vitro genotoxicity tests that come very close to human cells and have advantageous in vitro stability and metabolic functionality.

Especially with genotoxicity tests, the problem in the prior art has been that the metabolism of human hepatocytes is not sufficiently considered in cells lines and therefore the tested agents supply false positive, or even false, results in an in vitro testing environment.

Thus, the object of the present invention is to provide suitable hepatocytes for carrying out in vitro genotoxicity tests.

The object is achieved entirely by claim 1.

Proliferating hepatocytes surprisingly exhibit the following advantages:

The physiologically relevant properties of the proliferating hepatocytes according to the invention are specified in that these have at least four out of at least six different Phase I enzyme functions, even during the proliferative phase, preferably selected from the group consisting of CYP1A2, CYP2C9, CYP2C19, CYP2D6, CYP2E1 and CYP3A4, which are responsible for approximately 90% of all oxidative metabolization of drugs (Arimoto, 2006), and therefore in particular also contain more than 6 different Phase I enzymes, in particular ten different Phase I enzymes, preferably CYP1A1, CYP1A2, CYP2A6, CYP2B6, CYP2C8, CYP2C9, CYP2C19, CYP2D6, CYP2E1, CYP3A4, in particular thirteen different Phase I enzymes, in particular CYP1A1, CYP1A2, CYP2A6, CYP2B6, CYP2C8, CYP2C9, CYP2C19, CYP2D6, CYP2E1, CYP3A4, CYP3A5, CYP3A7, CYP4A11.

Additionally, the problem of false positive and false results from the prior art is also completely solved, because these proliferating hepatocytes:
a. have active Phase I and II activities during proliferation;
b. have significant activities that correspond to in vivo conditions in the enzyme provision;
c. have Phase I activities that are maintained over several days;
d. have enzyme activity that can be induced by way of reagents;
e. external metabolization by way of microsomes is consequently eliminated;
f. false positive results from reactive reagents that cannot penetrate cells are considerably reduced; and
g. after the agent has entered the cell, the agent itself and the resulting metabolite can act on the DNA, and consequently false negative results due to inadequate metabolization of the test substance are eliminated or considerably reduced.

The enrichment of such suitable hepatocytes is, for example, describe in the applicant's WO2009030217, which preferably can be obtained from primary cells. Moreover, proliferating hepatocytes can likewise be obtained from other precursor cells, such as stem cells, adult cells and other cells that can be differentiated.

Within the scope of the invention, the term “primary cells” shall be understood to mean explants that are obtained directly from bodily fluids or from bodily tissues of multicellular organisms, such as humans, mammals or suitable donors, and that have normal, which is to say not degenerated, cells. Primary cell cultures are primary cells that have been cultured up to the first passage. Primary cells have natural differentiation properties and are mortal.

So as to maintain cells in vitro, a method must be employed that compensates for the shortening of chromosomal telomeres that occurs with each cell division. One such option is the use of telomerase (Harley, C. B. and B. Villeponteau. 1995. Telomeres and telomerase in aging and cancer. Curr. Opin. Genet. Dev. 5:249-255). Cells that are able to compensate for the loss of telomeres, for example by way of telomerase, can grow an unlimited number of cell divisions and have immortality. However, over the course of the cell divisions, there is the inevitable drawback that mutations occur, which sooner or later must lead to the development of cancer.

So as to maintain human primary cells, or cells that can be differentiated, in vitro, the following steps can be carried out:

Primary cells or cells that can be differentiated are
a.) isolated;
b1.) functionally introduced into the cell with at least one proliferation gene or the gene product thereof; and/or
b2.) inactivated with at least one cellular factor that induces cell division arrest; and/or
b3.) transiently immortalized;
c.) cultured and/or passaged.

However, the starting material used is preferably human primary liver cells, which can be obtained by way of biopsy, for example.

Preferably, more than ten additional passages can be carried out as compared to primary cells, or more than 20 to 60 additional passages.

According to the invention, proliferating hepatocytes as described above are obtained, which are highly suited for carrying out genotoxicity tests. Particularly advantageously, cells can be obtained which do not take on any properties of tumor cells, and more particularly of malignant tumor cells, such as growth in soft agar or tumor growth in vivo (the growth of tumors in xenograft animal models).

Such cells are cultured on culture media that are known to a person skilled in the art.

Within the scope of the present invention, a proliferation gene is one that improves cell division and enables cell division capacity in the primary cell that can be increased to a limited extent, wherein the likelihood of cell transformation or changes of the differentiation properties is drastically reduced as compared to the cell lines from the prior art.

According to the invention, the proliferation gene is preferably selected from the group of viral proliferation genes: E6 and E7 of papillomaviruses such as HPV (human papillomavirus) and BPV (bovine papillomavirus); the large and small tumor antigens (TAgs) of polyomaviruses such as SV40, JK virus and BC virus; the E1A and E1B proteins of adenoviruses, EBNA proteins of the Epstein Barr virus (EBV); as well as the proliferation gene of HTLV and Herpesvirus saimiri and the respective coding proteins or the chimera thereof, selected from the group of the cellular proliferation genes, in particular from the following classes of genes: myc, jun, ras, src, fyg, myb, E2F and Mdm2 and TERT (a catalytic subunit of the enzyme telomerase), preferably human telomerase (hTERT).

However, according to the invention viral proliferation genes are preferred, with E6 and E7 of HPV or BPV being particularly preferred. To this end, proliferation genes of the HPV type can be used, which are related to malignant diseases. The best known examples of high-risk papillomaviruses are HPV16 and HPV18. Additional examples of the high-risk group include HPV 31, 33, 35, 39, 45, 51, 52, 56, 58, 59, 68, 73 and 82. However, it is also possible to use the E6 and E7 proliferation genes of so-called low-risk HPVs. Known examples include the HPV types 6 and 11, other HPV types of the low-risk group include HPV 40, 42, 43, 44, 54, 61, 70, 72 and 81. Moreover the corresponding chimera or chimeric gene products can be arbitrarily combined and used.

The significance of the E6 proteins in connection with an increase in proliferation lies above all in the inactivation of the p53 pathway and in the induction of telomerase. The significance of the E7 proteins in connection with an increase in proliferation lies above all in the inactivation of the pRb pathway. In connection with the invention, it is also possible to combine the proliferation genes of different serotypes of one virus species or of different virus species or even to produce and use chimeric proliferation genes of different serotypes of one virus species or different virus species. For example, one E6 domain in a chimeric gene can stem from HPV 16, for example, while another stems from HPV 6. Of course the proliferation genes can also be truncated or have one or more base exchanges, without departing from the scope of the invention. The aforementioned proliferation genes represent preferred embodiments and are not intended to limit the invention. The proliferation gene can optionally be the subject matter of a synthetic or artificially produced gene sequence.

These factors are “functionally introduced” into the target cells, the cell division capacity of which is supposed to be increased, and for this purpose the following gene transfer systems may be used, without being limited thereto: transfer of expression constructs of the above-mentioned gene functions into cells by way of the traditional calcium-phosphate method (Wigler, M. et al., 1977. Cell 11:223-232), by way of lipofection (Feigner, P. L. et al, 1987. Proc. Natl. Acad. Sci. U.S.A 84:7413-7417), by way of electroporation (Wolf, H. et al., 1994. Biophys. J. 66:524-531), by way of microinjection (Diacumakos, E. G. 1973. Methods Cell Biol. 7:287-311), by way of conjugates which are received via cellular receptors or receptor-independently. The above-mentioned gene functions can also be transferred to the target cells by way of viral vectors. Examples include retroviral vectors, AAV vectors, adenovirus vectors and HSV vectors, just to mention a few examples of vectors (overview of viral vectors in: Lundstrom, K. 2004. Technol. Cancer Res. Treat. 3:467-477; Robbins, P. D. and S. C. Ghivizzani. 1998. Pharmacol. Ther. 80:35-47). The term “functionally introduced” comprises in particular the transfection of the target cells by way of at least one proliferation gene.

The expression of the above-mentioned viral or cellular proliferation genes can be controlled by strong or weak constitutive promoters, tissue-specific promoters, or inducible promoters (Meyer-Ficca, M. L. et al. 2004. Anal. Biochem. 334:9-19), or the expression cassettes can be flanked by specific sequences for molecular excision systems. Examples include the Cre/Lox system (U.S. Pat. No. 4,959,317), the use of which leads to the molecular removal of the expression constructs from the genome of the target cells.

In a further embodiment, the gene products of the proliferation genes can likewise be functionally introduced directly into the target cell as such or by way of a fusion protein. Preferably these are messenger proteins (transport proteins) such as VP22, HIV TAT (Suzuki et al., 277 J. Biol. Chem. 2437-2443 2002 and Futaki 245 Int. J. polypeptide (WO97/12912 and WO99/11809) or Penetratin (Derossi et al., 8 Trends Cell Biol., 84-87 (1998), Engrailed (Gherbassi, D. & Simon, H. H. J. Neural Transm. Suppl 47-55 (2006), Morgan, R. 580 FEBS Lett., 2531-2533 (2006), Han, K. et al. 10 Mol. Cells 728-732 (2000)) or Hoxa-5 (Chatelin et al. 55 Mech. Dev. 111-117 (1996)), a polymer made of L-arginine or D-arginine amino acid residues (Can. Patent No. 2,094,658; U.S. Pat. No. 4,701,521; WO98/52614), a polymer made of L-lysine or D-lysine amino acid residues (Mai et al., 277 J. Biol. Chem. 30208-30218 (2002), Park et al. 13 Mol. Cells 202-208 (2002), Mi et al. 2 Mol. Ther. 339-347 (2000)), transcription factors such as BETA2/neuro D, PDX-1 (Noguchi and Matsumoto 60 Acta Med. Okayama 1-11, (2006), Noguchi et al. 52 Diabetes 1732-1737 (2003), Noguchi et al. 332 Biochem. Biophys. Res. Commun. 68-74 (2005)), nuclear localization signal, (Yoneda et al. 201 Exp. Cell Res. 313-320 (1992), histone-derived peptides (Lundberg and Johansson 291 Biochem. Biophys. Res. Comm. 367-371 (2002)), a polymer made of cationic macromolecules, FGF-1 and FGF-2, lactoferrin and the like, as described appropriately in the literature.

The invention therefore also relates to such proliferating hepatocytes which are transiently immortalized, preferably by way of i.) a polypeptide having cell immortalization activity;

ii.) a polypeptide that synthesizes telomeric DNA at chromosomal ends, or a respective fusion peptide thereof, wherein the fusion peptide in a first part consists of a transport protein, see above.

Such a polypeptide having cell immortalization activity can, for example, be obtained from the aforementioned viral or cellular proliferation genes. Moreover, reference is made to EP 1174436 B1 for the production of such polypeptides.

Such a polypeptide that synthesizes telomeric DNA at chromosomal ends is preferably selected from the group consisting of telomerase, telomerase reverse transcriptase (hTERT), p140, p105, p48 and p43. Moreover, reference is made to EP 1174436 B1 for the production of such polypeptides.

Within the scope of the present invention, “inactivated with at least one cellular factor that induces cell division arrest” shall be understood to mean that, for example, cell division arrest is activated as part of the senescence program (overview in: Ben Porath, I. and R. A. Weinberg. 2005. Int. J. Biochem. Cell Biol. 37:961-976.) or it refers to the cell division arrest that is activated in cells within the scope of the differentiation program. For example, it is known in the case of cardiac muscle cells that these stop dividing shortly after birth, which is regulated, among other things, by the expression of cell cycle inhibitors such as p16, p21, p27 (Brooks, G., et al. 1998. Cardiovasc. Res. 39, 301-311; Flink, I. L. et al., 1998. J. Mol. Cell Cardiol. 30, 563-578; Walsh, K. and Perlman, H. 1997. Curr. Opin. Genet. Dev. 7, 597-602). Similar processes surely apply to the majority of all primary cell types. Inactivation of cell cycle inhibitors in differentiated cells thus could cause the cells to return to proliferation. In the context of the invention, this also applies to further cell cycle inhibitory proteins not mentioned here.

Within the scope of the invention, the protein p53, which is important for controlling the cell cycle, and all proteins binding directly to p53, upstream and/or downstream factors of this p53 pathway can generally be inactivated so as to achieve the goal of increased cell division capacity (overview of the p53 pathway in: Giono, L. E. and J. J. Manfredi. 2006. J. Cell Physiol 209:13-20; Farid, N. R. 2004. Cancer Treat. Res. 122:149-164).

Within the scope of the invention, the protein P16/INK4a, which is important for controlling the cell cycle, and all proteins binding directly to P16/INK4a, upstream and/or downstream factors of this p16 pathway can generally be inactivated so as to achieve the goal of increased cell division capacity (overview of the p16/INK4a pathway in: Shapiro, G. I. et al., 2000. Cell Biochem. Biophys. 33:189-197).

Within the scope of the invention, the protein pRb, which is important for controlling the cell cycle, and all members of the pRb family (for example p107, p130) and all proteins binding directly to members of the pRb family, upstream and/or downstream factors of this pRb pathway can generally be inactivated so as to achieve the goal of increased cell division capacity (overview of the pRb pathway in: Godefroy, N. et al. 2006. Apoptosis. 11:659-661; Seville, L. L. et al. 2005. Curr. Cancer Drug Targets. 5:159-170).

Inactivation of cellular factors such as p53, pRb, p16 and the like can, for example, take place by the expression of dominant negative mutants of the corresponding factors (Herskowitz, I. 1987. Nature 329:219-222; Kupper, J. H., et al. 1995. Biochimie 77:450-455), by the inhibition of gene expression of these factors with the aid of antisense oligonucleotides (Zon, G. 1990. Ann. N.Y. Acad. Sci. 616:161-172), RNAi molecules (Aagaard, L. and J. J. Rossi. 2007. Adv. Drug Deliv. Rev. 59:75-86; Chakraborty, C. 2007. Curr. Drug Targets. 8:469-482), morpholinos (Angerer, L. M. and R. C. Angerer. 2004. Methods Cell Biol. 74:699-711), ribozymes (Sioud, M. and P. O. Iversen. 2005. Curr. Drug Targets. 6:647-653) or by way of gene knockout (Le, Y. and B. Sauer. 2000. Methods Mol. Biol. 136:477-485; Yamamura, K. 1999. Prog. Exp. Tumor Res. 35:13-24). These methods are known to a person skilled in the art and described in many places in literature. Inactivation can also take place by the action of specific antibodies (for example single chain antibodies, intrabodies and the like; overview in: Leath, C. A., III, et al. 2004. Int. J. Oncol. 24:765-771; Stocks, M. R. 2004. Drug Discov. Today 9:960-966). Inactivation can also take place by the use of chemical inhibitors of the cellular factors, for example by the use of kinase inhibitors.

One example of a kinase inhibitor is the substance imatinib (Gleevec®). A reduction in cell proliferation is achieved this way. Imatinib is a specific inhibitor that blocks the activity of Abl tyrosine kinase in diseased cells and thereby suppresses a pathologically increased proliferation of mutated blood stem cells.

In a preferred embodiment, the invention thus likewise relates to a method for producing an assay, comprising the following steps:

a.) providing a carrier material;
b.) immobilizing or fixing proliferating hepatocytes on this carrier material; and
bringing this cell from b.) in contact with an agent and determining the genotoxicity of the agent.

Within the scope of the present invention, an agent refers to any arbitrary substance, for example drugs and drug candidates that are approved or in development, and the precursors thereof; chemicals in general; biological active substances, which is to say molecules generated by cells, such as proteins that occur naturally this way, or in modified form in organisms or viruses, or can be formed there; including under physical effects such as electromagnetic radiation, heat, cold energy, sound or the like. An action of such an agent includes, but is not limited to, the generation of DNA damage such as nucleotide oxidation, deamination, loss of bases, strand breaks, adducts, DNA-DNA cross links. Agents that cause DNA to break are referred to as clastogenic. An indirect genotoxic effect of an agent is, for example, damage to the spindle apparatus, which is required for the segregation of chromosomes or sister chromatids, and wherein, due to the damage, for example, chromosome breaks or irregular chromosome distribution to the daughter cells during cell division may occur Agents that influence chromosome distribution are referred to as aneugenic. A change in the genetic makeup of a cell takes place as a result of these genotoxic effects of one or more agents, which can, but does not have to, be linked to direct toxicity that causes the cell to die. On the other hand, a change in the genetic makeup may manifest itself in changed gene activity, which results in a changed metabolism of the cell.

A positive event for determining genotoxicity can be proven in a broader sense with an assay reagent, for example by way of a fluorescence-labeled antibody or the like. In particular suitable bioanalytical methods should be mentioned here, for example immunohistochemistry, antibody arrays, Luminex/Luminol, ELISA, immunofluorescence, and radioimmunoassays.

The term “solid carrier” comprises embodiments such as a filter, a membrane, a magnetic spherule, a silicon wafer, glass, plastic material, metal, a chip, a mass spectrometry target, or a matrix, for example made of proteins, or other matrices, such as PEG for example, and the like

In a further preferred embodiment of the arrangement according to the invention (synonym: array), this array corresponds to a lattice having the size of a microtiter plate (96 wells, 384 wells or more), a silicon wafer, a chip, a mass spectrometry target or a matrix.

The carrier material (matrix) can be present in the form of spherical, non-aggregated particles, referred to as beads, fibers or a membrane, wherein the porosity of the matrix increases the surface. For example, the porosity can be increased in the customary manner by adding pore-forming material, such as cyclohexanol or 1-dodecanol, to the reaction mixture of the suspension polymerization.

### EXAMPLES

### Example 1

### Inducing the CYP3A4 Activity of Proliferating Hepatocytes

To begin with, hepatocytes that can proliferate are produced by treating primary human hepatocytes with the method described in WO 2009030217A2.

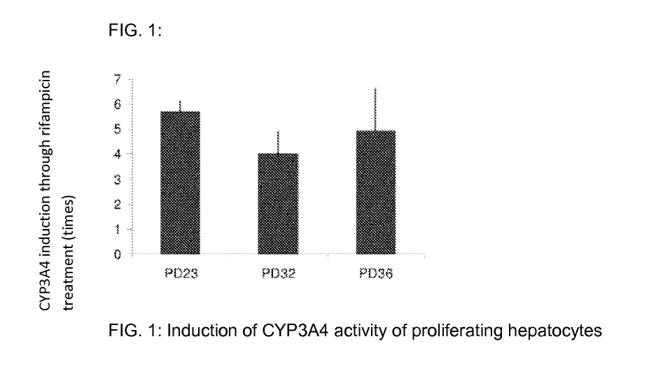

So as to analyze the metabolic capacity of the proliferating hepatocytes described in this invention, the induction of CYP3A4 activity was measured at various doubling figures (PD 23, 32 and 36). For this purpose, the cells were seeded on collagen-coated cell culture vessels in a density of 2.3×10⁴ cells/cm² and cultured for 4 days. Thereafter, the cells were treated every day for three days with rifampicin (20 μM), before measuring CYP3A4 activity by way of a luminescence-based P450-Glo assay (Promega). X times the induction (mean value±standard deviation, n=3) was calculated as the CYP3A4 activity (in RLU/s/well)) of the cells treated with rifampicin divided by the CYP3A4 activity of the control cells. It was possible to induce CYP3A4 activity of the proliferating hepatocytes in all three tested doubling periods (FIG. 1). The induction was similar for all doubling times that were analyzed and meets the criteria of the FDA for CYP3A4 induction in human hepatocytes (which is to say greater than four fold).

### Example 2

### Correctly Identifying Positive and Negative Substances for Genotoxicity Tests

So as to check the suitability of the proliferatable hepatocytes described in the patent with respect to genotoxicity tests, the cells were treated with substances that are considered positive and negative substances for genotoxicity. Genotoxicity was quantified by way of the share of induced micronuclei as compared to the control group using an FACS assay. In detail, the positive substances mitomycin C (MMC) and cyclophosphamide (CPA) and the negative substance curcumin were tested. CPA first has to be metabolized so as to have a genotoxic effect and is only detected in the currently used micronucleus test with V79 cells if the substance was previously converted with a CYP enzyme extract (S9 mix). In the standard genotoxicity tests that are based on rodent cell lines, curcumin is classified as being false positive.

The proliferating hepatocytes described in the invention were plated out in a density of 3000 cells/cm² on collagen-coated cell culture vessels and treated with the aforementioned substances in various concentrations. Within the scope of regulatory testing according to OECD, testing took place up to 50% cytotoxicity, which was determined for each substance beforehand by way of a MTT viability assay. Since at 48 hours the cell division speed of liver cells is less than that of the V79 cell line, longer incubation and recovery periods were selected for the treatments (indicated in each case).

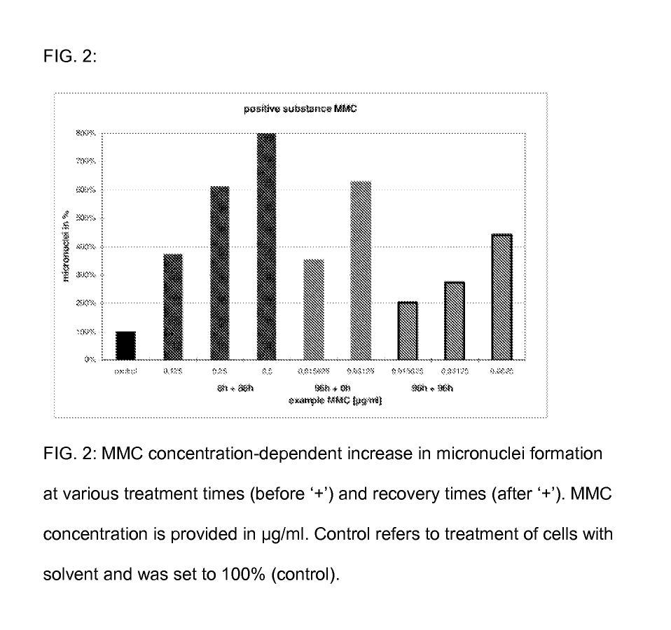

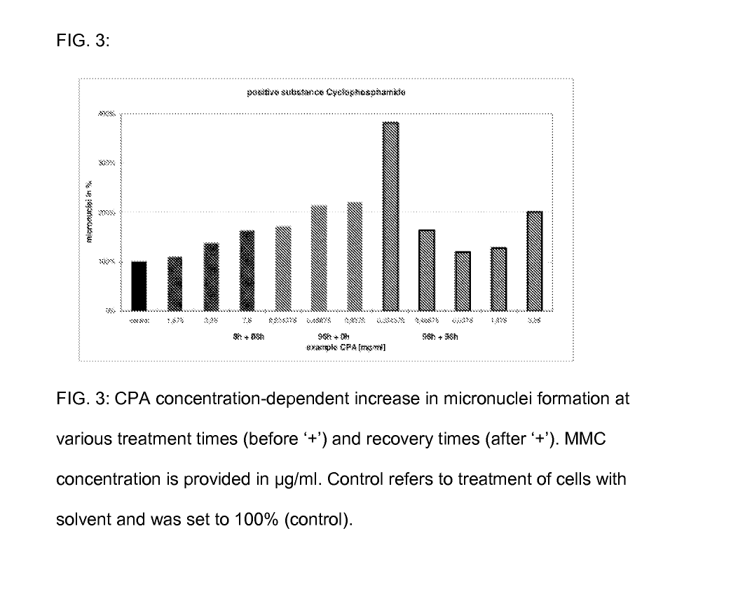

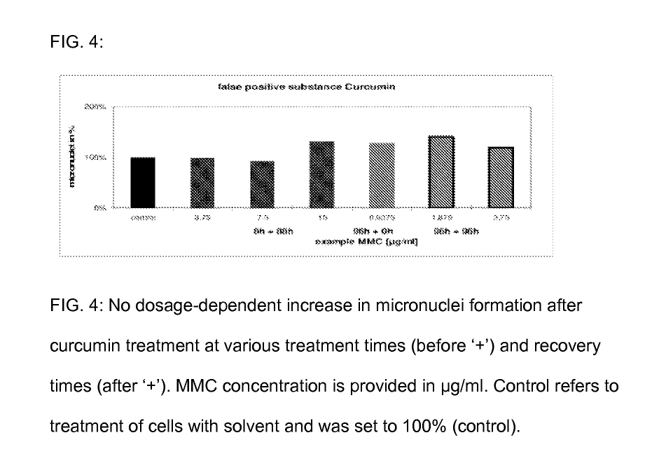

For MMC and CPA, a dose-effect relationship of the micronuclei formation was observed, so that these substances were clearly recognized as being positive (FIGS. 2 and 3). The correctly identified genotoxicity of CPA confirms the metabolic capacity of the cells In contrast, curcumin did not induce increased micronuclei formation and was therefore correctly classified as a negative substance (FIG. 4).

### BIBLIOGRAPHY

Agarwal, M. L., Taylor, W. R., Chernov, M. V., Chernova, O. B. and Stark, G. R. (1998).
Ames, B. N., Durston, W. E., Yamasaki, E., and Lee, F. D. (1973a). Carcinogens are mutagens: a simple test system combining liver homogenates for activation and bacteria for detection. Proc. Natl. Acad. Sci. U.S.A 70, 2281-2285.
Ames, B. N., Lee, F. D., and Durston, W. E. (1973b). An improved bacterial test system for the detection and classification of mutagens and carcinogens. Proc. Natl. Acad. Sci. U.S.A 70, 782-786.
Arimoto, R. (2006). Computational models for predicting interactions with cytochrome p450 enzyme. Curr. Top. Med. Chem. 6, 1609-1618.
Broschinski, L., Madie, S., and Hensel, C. (1998). Genotoxicity tests for new chemicals in Germany: routine in vitro test systems. Mutat. Res. 418, 121-129.
De Flora S., Russo, P., Pala, M., Fassina, G., Zunino, A., Bennicelli, C., Zanacchi, P., Camoirano, A., and Parodi, S. (1985). Assay of phenacetin genotoxicity using in vitro and in vivo test systems. J. Toxicol. Environ. Health 16, 355-377.
Elaut, G., Henkens, T., Papeleu, P., Snykers, S., Vinken, M., Vanhaecke, T., and Rogiers, V. (2006). Molecular mechanisms underlying the dedifferentiation process of isolated hepatocytes and their cultures. Curr Drug Metab 7, 629-660. Fenech, M. and Morley, A. A. (1985). Measurement of micronuclei in lymphocytes. Mutat. Res. 147, 29-36.
Gomez-Lechon, M. J., Donato, M. T., Castell, J. V., and Jover, R. (2004). Human hepatocytes in primary culture: the choice to investigate drug metabolism in man. Curr. Drug Metab 5, 443-462.
Hewitt et al (2007). Primary hepatocytes: current understanding of the regulation of metabolic enzymes and transporter proteins, and pharmaceutical practice for the use of hepatocytes in metabolism, enzyme induction, transporter, clearance, and hepatotoxicity studies. Drug Metab., 39, 159-234.
Kirkland D, Aardema M, Henderson L, Müller L. Evaluation of the ability of a battery of three in vitro genotoxicity tests to discriminate rodent carcinogens and non-carcinogens I. Sensitivity, specificity and relative predictivity. Mutat Res. 2005 Jul. 4; 584 (1-2):1-256.
Mathijs, K., Brauers, K. J., Jennen, D. G., Lizarraga, D., Kleinjans, J. C., and van Delft, J. H. (2010). Gene expression profiling in primary mouse hepatocytes discriminates true from false-positive genotoxic compounds. Mutagenesis.
Morita, T., Watanabe, Y., Takeda, K., and Okumura, K. (1989). Effects of pH in the in vitro chromosomal aberration test. Mutat. Res. 225, 55-60.
Rossi, D., Aiello, V., Mazzoni, L., Sensi, A., and Calzolari, E. (2007). In vitro short-term test evaluation of catecholestrogens genotoxicity. J. Steroid Biochem. Mol. Biol. 105, 98-105.
Singh, N. P., McCoy, M. T., Tice, R. R., and Schneider, E. L. (1988). A simple technique for quantitation of low levels of DNA damage in individual cells. Exp. Cell Res. 175, 184-191.

1. A method of testing genotoxicity in vitro, the method comprising using proliferating hepatocytes for carrying out the in vitro genotoxicity testing.

2. The method according to claim 1, further comprising carrying out the testing using the Ames test, chromosome aberration test, comet assay and micronucleus test.

3. The method according to claim 1, wherein the proliferating hepatocytes comprise at least four Phase I enzymes selected from the group consisting of CYP1A2, CYP2C9, CYP2C19, CYP2D6, CYP2E1, CYP3A4, CYP1A2, CYP2A6, CYP2B6, CYP2C8, CYP1A1, CYP3A5, CYP3A7 and CYP4A11.

4. The method according to claim 1, wherein the proliferating hepatocytes exhibit no viability in soft agar or no tumor growth in vivo.

5. The method according to claim 1, wherein the proliferating hepatocytes are from primary cells of humans or mammals and comprise at least one of
a) a proliferation gene, and
b) at least one cellular factor which is inactivated that induces cell division arrest, and
c) are transiently immortalized.

6. The method according to claim 5, wherein the cellular proliferation gene is selected from the group consisting of at least one of myc, jun, ras, src, fyg, myb, E2F, Mdm2, TERT; an E6 or E7 proliferation gene of papillomaviruses; a large or small tumor antigen of polyomaviruses; E1A or E1B protein of adenoviruses; an EBNA protein of the Epstein Barr virus; HTLV and Herpesvirus saimiri.

7. The method according to claim 5, wherein the viral proliferation gene is selected from the group consisting of at least one of E6 and E7 proliferating genes of HPV or BPV.

8. The method according to claim 5, wherein the cellular factor is selected from the group consisting of p53, p16, pRb, p107, p130 or the respective upstream or downstream factors thereof, or proteins binding thereto in the pathway, and the inactivation of the cellular factors takes place by way of the expression of dominant negative mutants or by the inhibition of gene expression of the cellular factors using antisense oligonucleotides, RNAi molecules, morpholinos, ribozymes, or by way of gene knockout, by the action of specific antibodies or by chemical inhibitors.

9. The method according to claim 5, wherein the transient immortalization takes place by way of a polypeptide having cell immortalization activity, a polypeptide that synthesizes telomeric DNA at chromosomal ends, or a respective fusion peptide thereof.

10. The method according to claim 5, wherein the transient immortalization takes place by way of a polypeptide having cell immortalization activity of an expression product of a cellular proliferation gene selected from the group consisting of myc, jun, ras, src, fyg, myb, E2F Mdm2 TERT; an E6 or E7 proliferation gene of papillomaviruses; a large or small tumor antigen of polyomaviruses; E1A or E1B protein of adenoviruses; an EBNA protein of the Epstein Barr virus; HTLV and Herpesvirus saimiri.

11. The method according to claim 5, wherein the transient immortalization takes place by way of a polypeptide that synthesizes telomeric DNA at chromosomal ends which is selected from the group consisting of telomerase, telomerase reverse transcriptase (hTERT), P140, P105, p48 and p43.

12. The method according to claim 5, wherein the transient immortalization takes place by way of a fusion peptide, wherein a first part is a transport polypeptide, and a second part is a polypeptide that is an expression product of a cellular proliferation gene selected from the group consisting of myc, jun, ras, src, fyg, myb, E2F, Mdm2, TERT; an E6 or E7 proliferation gene of papillomaviruses; a large or small tumor antigen of polyomaviruses; E1A or E1B protein of adenoviruses; an EBNA protein of the Epstein Barr virus; HTLV and Herpesvirus saimiri.

13. A method for producing an assay, comprising the following steps:
a) providing a carrier material;
b) immobilizing or fixing proliferating hepatocytes on the carrier material;
c) bringing the immobilized or fixed proliferating hepatocytes on the carrier from b) in contact with an agent; and
d) determining the genotoxicity of the agent.

14. A method for producing an assay according to claim 13, wherein the agent is selected from the group of chemical and biological active substances, drugs and cosmetics.

15. The method according to claim 5, wherein the transient immortalization takes place by way of a polypeptide having cell immortalization activity selected from the group of an expression product of a viral proliferation gene selected from the group consisting of at least one of E6 and E7 proliferating genes of HPV or BPV.

16. The method according to claim 5, wherein the transient immortalization takes place by way of a fusion peptide, wherein a first part is a transport polypeptide, a second part is a polypeptide that is an expression product of a viral proliferation gene selected from the group consisting of at least one of E6 and E7 proliferating genes of HPV or BPV.

17. The method according to claim 12, wherein the transport polypeptide is selected from the group consisting of at least one of VP22, HIV TAT, (HIV) REV, Antennapedia polypeptide, Penetratin, Engrailed, Hoxa-5, a polymer made of L-arginine or D-arginine amino acid residues, a polymer made of L-lysine or D-lysine amino acid residues, a transcription factor, nuclear localization signal, histone-derived peptides, a polymer made of cationic macromolecules, FGF-1, FGF-2, and lactoferrin.

18. The method according to claim 17, wherein the transcription factor is selected from the group consisting of BETA2/neuro D and PDX-1.

19. The method according to claim 5, wherein the proliferation gene is at least one of a cellular proliferation gene and a viral proliferation gene.

20. The method according to claim 6, wherein the viral proliferation gene selected from the group E6 or E7 proliferating gene of papillomaviruses is HPV; and the large or small tumor antigen of polyomaviruses is selected from the group consisting of SV40, JK virus and BC virus.

21. The method according to claim 7, wherein the E6 or E7 proliferating gene of HPV is selected from the group consisting of at least one of HPV6, HPV11, HPV16, HPV 18, HPV31, HPV33, HPV35, HPV39, HPV40, HPV42, HPV43, HPV44, HPV45, HPV51, HPV52, HPV54, HPV56, HPV58, HPV59, HPV61, HPV68, HPV70, HPV72, HPV73, HPV81 and HPV82.

22. The method according to claim 12, wherein the transport polypeptide is selected from the group consisting of at least one of VP22, HIV TAT, (HIV) REV, Antennapedia polypeptide, Penetratin, Engrailed, Hoxa-5, a polymer made of L-arginine or D-arginine amino acid residues, a polymer made of L-lysine or D-lysine amino acid residues, a transcription factor, nuclear localization signal, histone-derived peptides, a polymer made of cationic macromolecules, FGF-1, FGF-2, and lactoferrin.

23. The method according to claim 22, wherein the transcription factor is selected from the group consisting of BETA2/neuro D and PDX-1.
